(19) United States
(12) Patent Application Publication
(10) Pub. No.: US 2015/0342717 A1
(43) Pub. Date: Dec. 3, 2015

(54) TEMPORARY VALVE AND FILTER ON GUIDE CATHETER

(71) Applicants: Michael J. O'DONNELL, Ann Arbor, MI (US); Brian A. BIANCUCCI, Chelsea, MI (US)

(72) Inventors: Michael J. O'DONNELL, Ann Arbor, MI (US); Brian A. BIANCUCCI, Chelsea, MI (US)

(21) Appl. No.: 14/727,042

(22) Filed: Jun. 1, 2015

Related U.S. Application Data
(60) Provisional application No. 62/005,322, filed on May 30, 2014.

(51) Int. Cl. A61F 2/01; A61F 2/962; A61F 2/966; A61F 2/24

(52) U.S. Cl. CPC A61F 2/013; A61F 2/24; A61F 2/2439; A61F 2/962; A61F 2/966; A61F 2/2418; A61F 2/2412; A61M 2205/75; A61M 2025/0098

(57) ABSTRACT

A temporary valve and a filtration device can be added to a delivery catheter or guide catheter to improve hemodynamics and provide embolic protection. Both the valve and filter can mount onto the outer diameter of a delivery or guide catheter, which offers several distinct advantages. First, the inner lumen of the delivery or guide catheter remains unaffected, thereby allowing the standard procedure steps to take place without any changes or interruptions, or with minimal changes or interruptions.

### CROSS REFERENCE TO RELATED APPLICATION

This application claims priority to U.S. Provisional Patent Application No. 62/005,322, filed May 30, 2014, titled “TEMPORARY VALVE AND FILTER ON GUIDE CATHETER,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This invention relates generally to systems and methods used in cardiovascular procedures, and more specifically to systems and methods for providing a temporary valve and/or filter during a cardiovascular procedure.

### BACKGROUND

Heart valves, such as the aortic valve, are sometimes damaged by diseases or by aging which can cause problems with the proper function of the valve. Valve replacement may be required in severe cases to restore cardiac function. Catheter based valve replacement has been proposed as a way to effect valve replacement percutaneously and to avoid open-heart surgery. Such procedures involve excision of the native valve and replacement of the native valve with a prosthetic valve, or installation of a prosthetic valve over the native valve. To avoid cardiopulmonary bypass, the catheter based valve replacement is performed on a beating heart. Following excision of the native valve, no valve is present to preserve the pumping action of the heart while the permanent prosthetic valve is being implanted.

Transcatheter aortic valve replacement (TAVR) is minimally invasive surgical procedure that repairs the valve without removing the old, damaged valve. Instead, it wedges a replacement valve into the aortic valve's place. The surgery may be called a transcatheter aortic valve replacement (TAVR) or transcatheter aortic valve implantation (TAVI). However, in some cases it may be beneficial to remove a damaged cardiac (e.g., aortic) valve prior to implantation of the new replacement valve. Even in situations where the valve is not removed (traditional TAVR) it would be beneficial to minimize or reduce the negative effects associated with disruption of cardiac valves. Often this means that the valvular procedure is performed as quickly as possible, and in addition, a temporary pacemaker is often used to force the patient's heart to beat at an abnormally high rate, in an attempt to minimize blood flow and leaflet motion.

Use of a temporary valve during such procedures would enhance hemodynamic stability by preserving normal or near normal cardiac flow and pressures. As a result, patient safety would be enhanced and the quality of the procedure would be improved by offering the physician more time and control.

In addition to TAVR and similar procedures, other interventional cardiac procedures require placement of catheters and other devices through the valves of the heart. Disruption of the normal flow control function of a valve can result from such procedures, causing leakage and hemodynamic instability. Another complication of catheter interventions is the potential for disruption of atherosclerotic plaques and the dislodgement of calcific nodules on heart valves, which can cause debris to enter the blood stream. The debris generated in this manner may in turn cause strokes by occluding small arteries and blocking blood flow to distal brain tissue. Currently, catheter-delivered filtration devices are used to minimize and/or reduce these events.

Existing catheter-based temporary valves and filtration devices are delivered as independent devices, requiring separate insertion steps and may also interfere with the primary delivery catheter.

Thus, it would be beneficial to provide one or more apparatuses and methods that may provide a temporary valve, a working channel through the valve, and/or a filter that may be used concurrently with a temporary valve.

### SUMMARY OF THE DISCLOSURE

Described herein are temporary valve and a filtration apparatuses (e.g., devices and systems) that may be coupled directly onto the delivery catheter or guide catheter. The valve and filter may be integrated (e.g., nested) together or separate, and may both be collapsed onto the outside of the catheter. Both the filter and the valve may mount onto the outer diameter of a delivery or guide catheter, which offers several distinct advantages. First, the inner lumen of the delivery or guide catheter remains unaffected, thereby allowing the standard procedure steps to take place without any changes or interruptions, or with minimal changes or interruptions. Second, the need for separate devices and procedures (e.g., inserting and placing an embolic filter catheter in a distal location) is eliminated, which reduces risk, complexity and cost. The temporary valve (which may be referred to as a parachute filter or a strutless or unsupported filter) may be used with or without the filter. Alternately, the filters described herein may be used without the temporary valves described herein.

The temporary valves described herein have many advantages over other catheter-mounted valves, including in particular the use of a soft and/or compliant material to form the body of the temporary valve, which does not require a separate support, such as a strut, wire, band, etc. to maintain its shape and function. Such strutless constructions (particularly in the distal end region which occludes blood flow when expanded) may allow the device to be positioned within a vessel, and expand and contract during pumping of the blood, with minimal risk of damage to vessels, and surprisingly allows a robust response to changing blood flow.

For example a temporary valve apparatus (e.g., system or device) may include: a guide catheter having an inner diameter and an outer diameter and a proximal end and a distal end; and a valve mounted around an outer distal portion of the guide catheter, the valve made of a thin and flexible material and comprising a conical distal section and a conical proximal section, the conical distal section of the valve tapering towards the distal end of the guide catheter and the conical proximal section of the valve tapering towards the proximal end of the guide catheter, the conical proximal section of the valve having a plurality of openings, the valve configured to adopt a collapsed configuration against the guide catheter when fluid flows over the valve from the distal end of the guide catheter to the proximal end of the guide catheter, the valve configured to adopt an expanded configuration when fluid flows over the valve from the proximal end of the guide catheter to the distal end of the guide catheter.

Any of the temporary valve apparatuses described herein may include a filter disposed on the guide catheter along with the temporary valve. For example, the filter may be positioned proximally to the distal section of the valve, and configured to remain in an open configuration when the valve is in either the expanded configuration or the collapsed configuration.

In any of these variations, the temporary valve may comprise a one-piece construction, with no rigid components. As will be described in greater detail below, both sections (the distal and proximal sections) may be continuous. The distal section may include an unbroken (without holes or openings) sheet of material that forms a barrier to the blood when expanded (opened). The proximal section typically includes large (e.g., about 50%, 60%, 70%, 80%, 90%, etc. or more of the surface area of the proximal section) openings or windows.

As mentioned, the temporary valve may be “strutless” meaning that it does not include a support strut or structure. In some variations both the proximal section and the distal section are not supported (are unsupported) by any strut. In some variations the proximal portion but not the distal portion is supported by one or more struts.

The filter may also be conically shaped, or bell-shaped, and communicate with the temporary valve, such as the proximal portion of the temporary valve. In some variations the filter is bell-shaped. The filter may be concentrically positioned around the temporary valve (e.g., the proximal portion of the temporary valve). For example, the filter and valve are nested together.

The filter may include a support structure configured to support the filter in the open configuration. For example, the support structure may comprise one or more of: a ring, a spiral strut, and/or a plurality of struts. The support structure may be made of a metal (such as a shape memory metal, e.g. Nitinol), or a polymeric material.

The filter may be fixed to the outer diameter of the catheter, or it may be slidably disposed on the guide catheter.

Any of the apparatuses described herein may include a sheath that is slidably disposed over the guide catheter, valve, and/or filter. The sheath may collapse the temporary valve and/or filter so that it can be positioned or removed. Alternatively or additionally, the filter may include one or more control wires that extend to the proximal end of the guide catheter, the one or more control wires configured to collapse and/or expand the filter. In variations in which the filter may be concentrically positioned, e.g., over all a portion (such as the proximal portion) of the temporary valve, collapsing the filter may collapse the temporary valve, and allow it to be removed, positioned and/or repositioned.

In general, the distal section and the proximal section are conical, meaning that they have a generally round transverse cross-section; as used herein a conical shape may include generally oval (e.g. not limited to strictly circular) cross-sections. The conical shape of the distal and proximal sections generally refers to the shape in the expanded configuration. These conical shapes refer to the general, overall, shape of these sections. These shapes are not intended to refer to strict geometric interpretations of the conical shape, as they may be somewhat curved (e.g., a longitudinal section along the outer edge may not be perfectly straight, but may curve or bow outwards slightly).

Either or both the proximal and the distal sections of the valve may be attached to the guide catheter with sleeve, such as a distal end sleeve at the distal end and a proximal end sleeve at the proximal end. The distal end sleeve and proximal end sleeve may have an inner diameter that matches the outer diameter of the guide catheter.

In general, the proximal section of the valve may be longer than the distal section of the valve. For example, the proximal section of the valve may be about twice as long as the distal section of the valve. Alternatively or additionally, the proximal section of the valve may be tapered at an angle between about 30 to 60 percent of the angle of the taper of the distal section of the valve (e.g., about 05% of the angle of taper of the distal section).

In some variation one or both ends (end sleeves) of the valve are attached to the catheter. Alternatively, one or both ends (end sleeves) may be slidably disposed on the guide catheter. Thus, the entire temporary valve may be slideable on the catheter; the catheter may include one or more stops to prevent the valve from sliding (e.g., distally) off of the catheter.

In some variation the temporary valve and filter may be supported by a common frame, the common frame comprising a first plurality of struts for supporting the filter and a second plurality of struts for supporting the valve. The first plurality of struts for supporting the filter may have free ends that are configured to open up to an expanded configuration, and/or the second plurality of struts for supporting the valve may have ends that remain proximate the guide catheter.

For example, a temporary valve system may include: a guide catheter having an inner diameter and an outer diameter and a proximal end and a distal end; and a valve mounted around an outer distal portion of the guide catheter, the valve made of a thin and flexible material and comprising a conical distal section and a conical proximal section, wherein the conical distal section of the valve is unsupported by struts and tapers towards the distal end of the guide catheter, and wherein the conical proximal section of the valve tapers towards the proximal end of the guide catheter, the conical proximal section of the valve having between about 1 and 5 openings (e.g., 3), the valve configured to adopt a collapsed configuration against the guide catheter when fluid flows over the valve from the distal end of the guide catheter to the proximal end of the guide catheter, the valve configured to adopt an expanded configuration when fluid flows over the valve from the proximal end of the guide catheter to the distal end of the guide catheter; and a filter disposed on the guide catheter proximally to the distal section of the valve, the filter configured to remain in an open configuration when the valve is in either the expanded configuration or the collapsed configuration.

In general, also described herein are methods of using any of the apparatuses described herein. For example, described herein are methods of providing a temporary valve to a blood vessel, the method comprising: inserting a guide catheter into the blood vessel, the guide catheter having a valve mounted on an outer distal portion of the guide catheter, the valve made of a thin and flexible material and comprising a conical distal section and a conical proximal section, wherein the conical distal section is unsupported by struts and tapers towards the distal end of the guide catheter and wherein the conical proximal section of the valve tapers towards the proximal end of the guide catheter, the proximal section of the valve having a plurality of openings, the valve configured to adopt a collapsed configuration against the catheter when a fluid flows over the valve from the distal end of the guide catheter to the proximal end of the guide catheter, the valve configured to adopt an expanded configuration when the fluid flows over the valve from the proximal end of the guide catheter to the distal end of the guide catheter; advancing the distal end of the guide catheter to the ascending aorta; positioning the valve in the aorta; and deploying the valve.

Any of these methods may also or alternatively include filtering blood passing past the guide catheter with a filter disposed on the guide catheter proximally to the distal section of the valve, the filter configured to remain in an open configuration when the valve is in either the expanded configuration or the collapsed configuration.

In some variations the method may include positioning the filter between the aortic root and brachiocephalic trunk.

Deploying the valve may include retracting a sleeve disposed over the guide catheter and the valve.

Any of these methods may include collapsing the valve by advancing a sleeve over the guide catheter and the valve.

Positioning the valve may include positioning the valve between the aortic root and the brachiocephalic trunk. Positioning the valve further comprises positioning the valve in the descending aorta.

The filter may be deployed, for example, by manipulating one or more control wires to deploy the filter.

Any of the methods described herein may also include operating though the catheter (e.g., before, during or after deploying the temporary valve). For example, the method may include deploying an excision device through the catheter and removing a cardiac valve, and/or deploying a replacement valve through the catheter.

As mentioned above, any of the apparatuses described herein may include just a filter, without requiring a temporary valve. For example, a temporary filter system may include: a guide catheter having an inner diameter and an outer diameter and a proximal end and a distal end; a filter mounted around an outer distal portion of the guide catheter; a sheath that is slidably disposed over the guide catheter and configured to slide distally to collapse the filter into a closed configuration and slide proximally to allow the filter to expand into an open configuration around the outer diameter of the guide catheter; and a support structure configured to support the valve in the open configuration. The filter may be configured as described above.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1C illustrate various views of one embodiment of a temporary valve.

FIGS. 2A and 2B illustrate cross-sectional views of an embodiment of a temporary valve disposed in a vessel during forward flow and backflow conditions.

FIGS. 3A-3C illustrate various embodiments of a filter mounted to a catheter with a retractable sheath for collapsing and deploying the filter.

FIGS. 4A-4C illustrate an embodiment of the filter and valve in combination.

FIG. 5 illustrates an embodiment of a valve nested with a filter in the ascending aorta.

FIGS. 6A and 6B illustrate an embodiment of a frame that can be used to support both a valve and a filter.

FIGS. 7A and 7B illustrate bench testing data of an embodiment of the temporary valve.

FIG. 8 illustrates one method of removing an existing valve (e.g., aortic valve) using the apparatus as described herein.

FIG. 9 is a schematic illustrating one method of operating a temporary valve apparatus as described herein.

FIG. 10 shows one variation of a frame supporting a filter that may be used with a temporary valve or without a temporary valve.

### DETAILED DESCRIPTION

Described herein are temporary flow control valves and filters that can be secured to the outer diameter of a delivery or guide catheter.

### Temporary Valve:

In some embodiments, the valve can be made from any thin, flexible material appropriate for human blood contact such as those used in medical balloons (e.g., urethane, silicone, nylon, latex). In some embodiments, the valve is of one-piece construction, with no rigid struts, frame, components or structure. In some embodiments, the valve is constructed of a non-compliant or weakly compliant material that exhibits little elasticity.

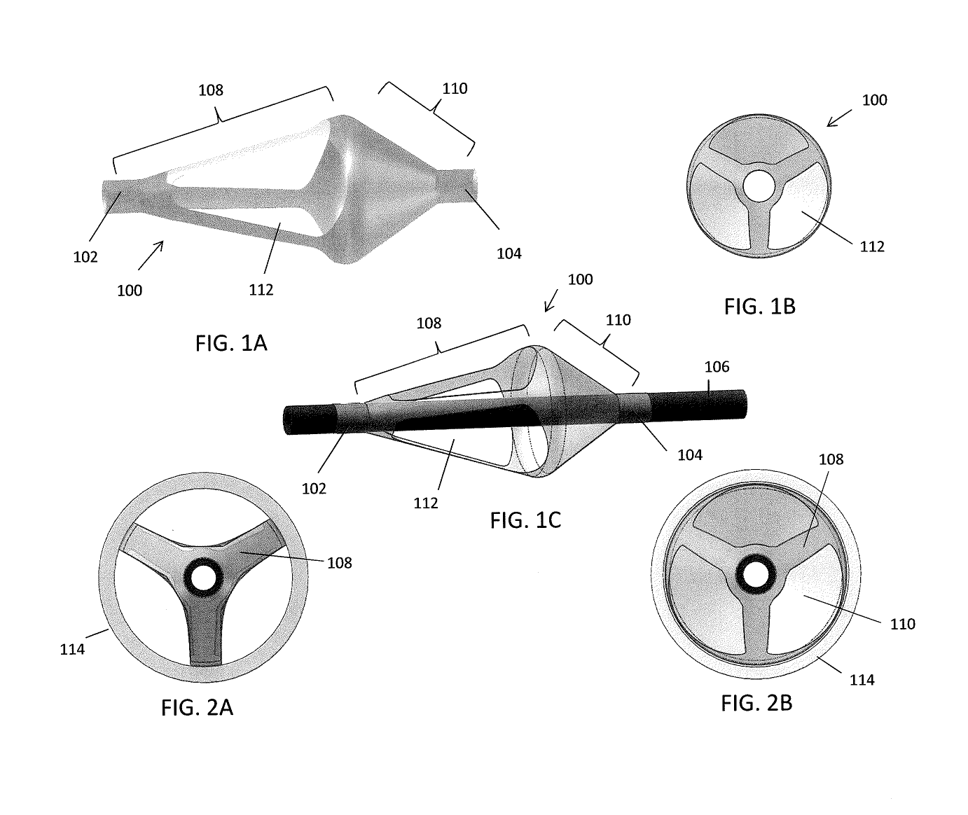

For example, as shown in FIGS. 1A-1C, the valve 100 has two end sleeves 102, 104 which have a diameter matching the outer diameter of the catheter 106 it is to be used with. In some embodiments, the valve 100 can be disposed concentrically around the catheter 106. The valve body has two sections of unequal length and taper. In some embodiments, the proximal (downstream) section 108 is generally longer than the distal (upstream) section 110 and is tapered at an angle about half that of the proximal section 108. In other embodiments, the proximal section 108 is tapered at an angle about 30 to 60 percent of the angle of the taper of the distal section 110 of the valve. In some embodiments, the proximal section 108 is between about 50 percent to 300 percent the length of the distal section 110. In some embodiments, the proximal section 108 is about twice the length of the distal section 110. The terms “proximal” and “distal” refer to the location of a feature in relation to the user holding the device, where a feature closer to the user is “proximal” and a feature further away from the user is “distal.” The terms “downstream” and “upstream” refer to a location in reference to the direction of blood flow. The maximum diameter of the valve body, which is typically located at the interface between the proximal section 108 and the distal section 110, is chosen to be as large as or slightly larger than the diameter of the vessel in which it is placed, such as the ascending aorta. The valve can be manufactured in multiple sizes as appropriate.

The proximal section 108 has multiple openings 112, preferably three, which allows the valve 100 to more closely mimic the performance of a three leaflet valve, such as the aortic valve. In other embodiments, the proximal section 108 may have two openings, or more than three openings. As shown in FIGS. 2A and 2B, during forward flow in a vessel 114 the valve material of the distal section 110 collapses to allow flow to pass by. When the flow reverses, the fluid is halted in the distal pocket area as the distal section 110 expands, preventing reverse flow.

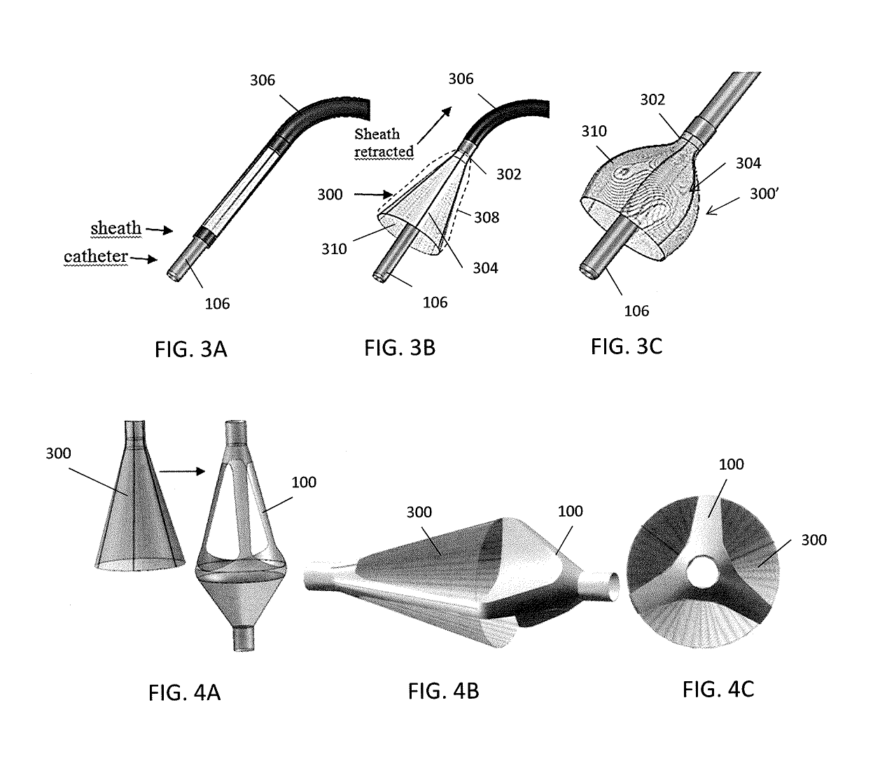

In some embodiments, the valve can be folded or otherwise collapsed around the catheter so that it can be covered with a sheath for delivery and retrieval. During positioning and removal of the catheter, the sheath can be advanced over the catheter to cover the valve. When the valve is needed, the valve can be deployed by retracting the sheath from the valve. FIG. 3A illustrates an example of a sheath that can be advanced and retracted over the catheter to collapse or deploy a filter, as further described below. This same catheter and sheath configuration can also be used to collapse and deploy a valve.

### Filter:

In some embodiments as shown in FIGS. 3A-3C, the filter 300 has a sleeve 302 on one end used for mounting to the outer diameter of the catheter 106. The other end of the filter 300 opens to a diameter large enough to occupy the cross section of the vessel, such as the ascending aorta.

In some embodiments, rigid supports 304 run along the length of the filter 300 and hold it in the open configuration. The supports 304 can form a frame 305. The supports 304 may be made from a thin metal or a shape memory metal (e.g., Nitinol). The supports 304 can flex to allow the filter 300 to be collapsed against the body of the catheter 106 and covered with a sheath 306. Retracting the sheath 306 will cause the filter 300 to expand to its open configuration. Alternatively, the filter expansion may be controlled by wires 308 attached to the open end of the filter 300, shown in dotted lines in FIG. 3B, which run along the length of the catheter. The wires 308 can be controlled by the operator at the proximal end of the catheter 106. For example, the operator can retract or pull the wires 308 to expand the filter 300, and advance or push the wires 308 to collapse the filter 300. In some embodiments, the rigid supports 304 may be linear and create a conical shaped filter 300 or have curvature and create a bell-shaped filter 300′. In some embodiments of the bell-shaped filter 300′, the maximum diameter of the filter is offset from the filter opening.

Surrounding the rigid supports 304 is a thin porous material or membrane 310 that allows blood flow to pass through but blocks passage of large particulates. The pores may be any appropriate size (e.g., between about 30, 35, 40, 45, 50, 55, 60, 65, 70, 75, etc. microns and about 75, 80, 85, 90, 95, 100, 110, 120, 130, 140, 150, 160, 170, etc. microns). In some embodiments, the filter membrane 310 can have a pore size between about 55 to 80 microns. In general, the filter may have a pore size range of between about 50 to about 150 microns. For example, FIG. 10 shows another example of a filter 300 configured as a mesh/membrane having a pore size of between about 50 and 150 microns (e.g., between 55-80 microns, etc.) and is configured as a collapsible/expandable filter having a Nitinol frame 304 covered with filter material and mounted on tubing 355.

In some embodiments, the filter 300 can be advanced to and deployed at a location in the ascending aorta that is in between the aortic root and brachiocephalic trunk in order to provide embolic protection to the carotid arteries that supply blood to the brain.

### Combined Valve and Filter:

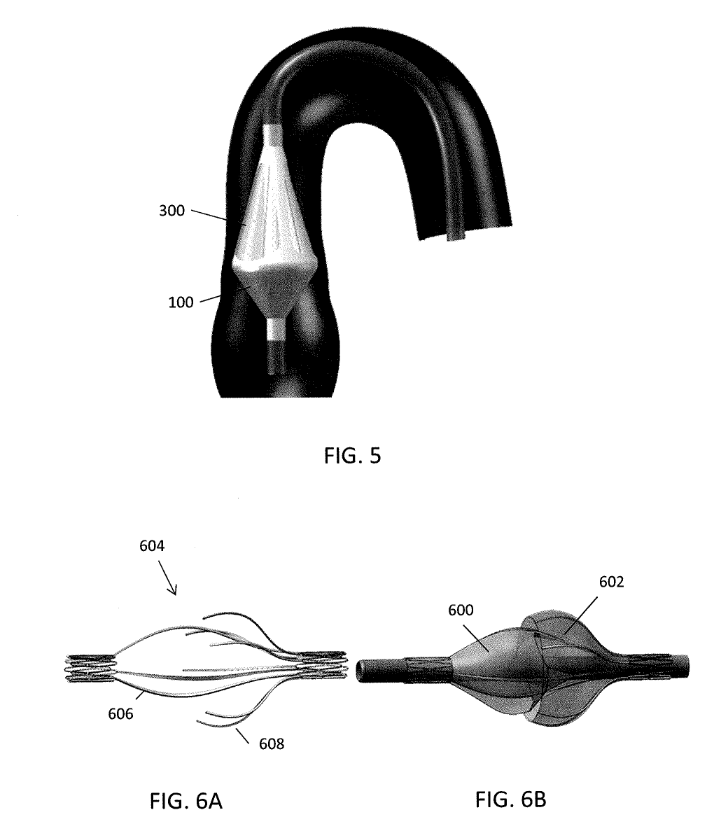

As shown in FIGS. 4A-4C, the valve 100 and filter 300 can be integrated into a nested configuration by inserting the filter 300 onto the outflow portion of the valve 100. This configuration reduces the amount of space the two components would otherwise occupy separately. When nested, the filter frame 305 can continue to keep the filter 300 expanded while the valve 100 is free to independently open and close. In some embodiments, the size and shape of the filter 300 can match or be complementary to that of the proximal section 108 of the valve 100 in order to facilitate nesting. FIG. 5 illustrates the valve 100 and filter 300 nested together in the ascending aorta.

In some embodiments, the filter 300 can be positioned separately from the valve 100. For example, in some embodiments, the filter 300 can be located proximally or downstream the valve 100. In other embodiments, the filter 300 can be located distally or upstream the valve 100, such as when the filter 300 is located in the ascending aorta between the aortic root and brachiocephalic trunk while the valve 100 is positioned in the descending aorta.

As shown in FIGS. 6A and 6B, the valve 600 and filter 602 may also be combined with a common frame 604 which supports both of them. In some embodiments, the frame 604 can have a set of struts 606 that support the temporary valve 600, while a separate set of struts 608 support the filter 602. In other embodiments, the frame can have a set of struts that supports both the valve and the filter.

The valve and filter can be used with or without each other. For example, a delivery catheter may have both devices nested together, may have just the temporary valve, or may have just the filter.

In some embodiments, the valve and filter can be secured to the outer diameter of the catheter (e.g., by bonding) so that they are fixed in place relative to the tip of the catheter. Alternatively, the mounting sleeves of each device may slide over the outer diameter of the catheter so that they can be introduced and positioned on the catheter as needed by the operator. In some embodiments, the mounting sleeves of each device can be attached to control wires which allow the operator advance and retract the devices over the catheter.

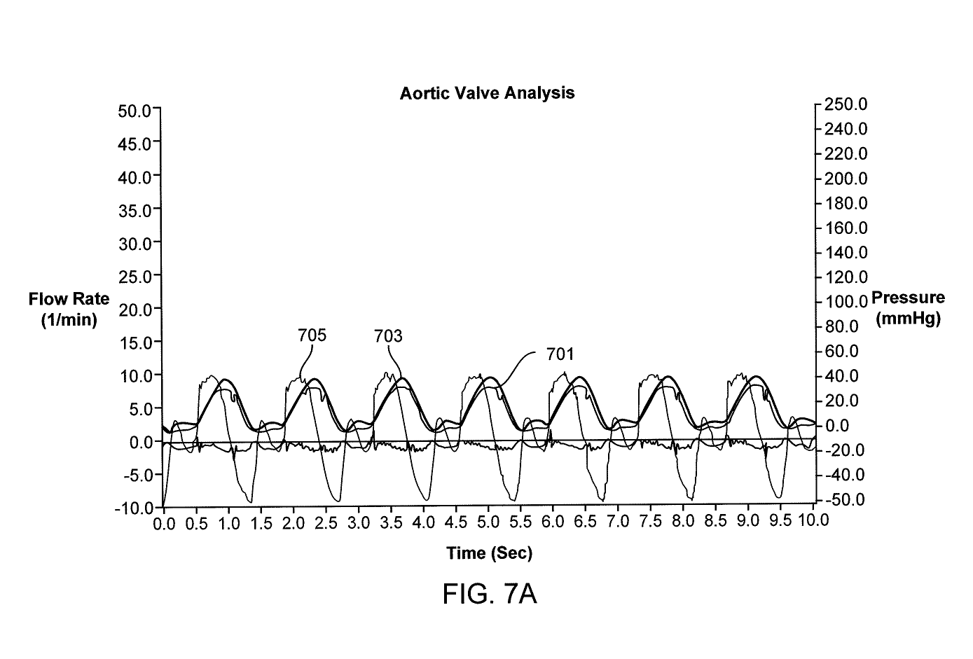

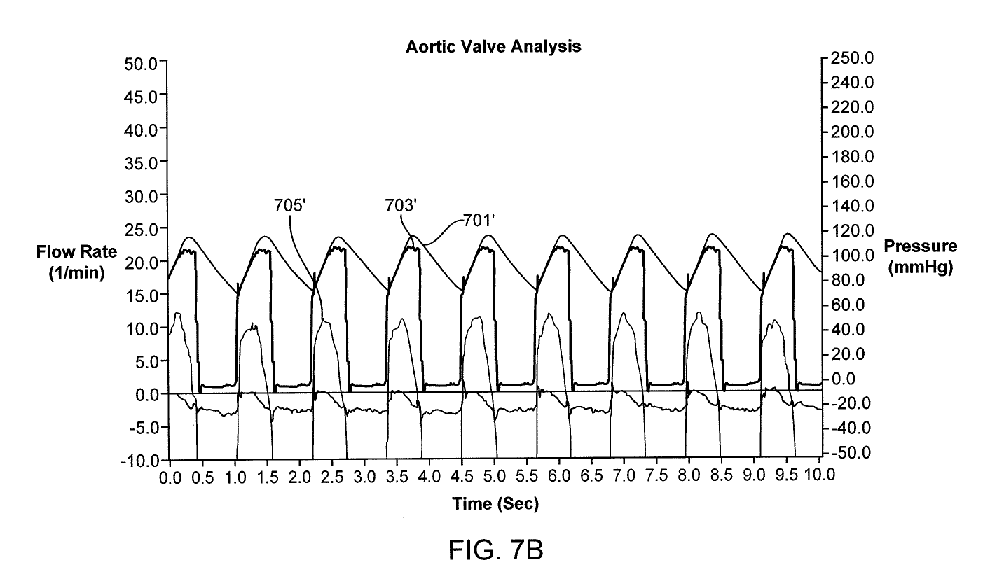

FIGS. 7A and 7B illustrate bench data from testing the temporary valve in a model with no aortic valve. FIG. 7A illustrates aortic flow without the aortic valve and without a temporary valve. FIG. 7B illustrates aortic flow without the aortic valve but with the addition of the temporary valve. The data shows that addition of the temporary valve greatly reduces the amount of backflow or retrograde flow, which is shown in the graphs as negative flow values, and therefore restores a more normal hemodynamic flow pattern. For example, in FIG. 7A, the blood pressure 701 is very low (between about 10 mmHg and 50 mmHg); with the temporary valve in position, a more normal blood pressure 701′ is produced, as shown in FIG. 7B, which shows a blood pressure of between about 80 mmHg and 120 mmHg (120/80). Similarly, the ventral pressure 703 without the valve is restored more physiologically normal levels 703′ when the temporary valve is used. As expected when no valve is present, the flow rate 705 through the model aortic region is both positive and negative, but is primarily positive-going 705′ (with a slight backflow) with the temporary valve in position. Thus, a model temporary valve as described herein may restore physiologically acceptable parameters when operated even with a normal (e.g., aortic) valve removed.

In general, the filter and valves described herein (“temporary valves”) can be used in various cardiovascular procedures, such as when embolic protection is desired and/or where the aortic valve is displaced or compromised. For example, the filter and valve described herein can be used in a transcatheter aortic valve replacement (TAVR) procedure. During a traditional TAVR procedure, a valve is placed inside the native calcified valve, which reduces the orifice area, creates potential embolic material, creates potential paravalvular leaks, and can induce conduction abnormalities. A separate downstream filter is usually needed to protect against cerebral and other emboli, and rapid pacing is also usually needed.

Using the filter and valve disclosed herein allows the TAVR procedure to be improved. Once the valve and filter are in place, the native valve can be removed, if desired, instead of being left in place. Placing the temporary valve in the ascending aorta allows hemodynamic stability to be maintained, even if the aortic valve is removed. The filter additionally provides embolic protection while the procedure is performed. Since the valve and filter are integrated with the guide catheter, only a single guide catheter is needed while maintaining the lumen of the guide catheter for the catheters used to deliver the new valve. The temporary valve and filter described herein can be used with any type of TAVR valve.

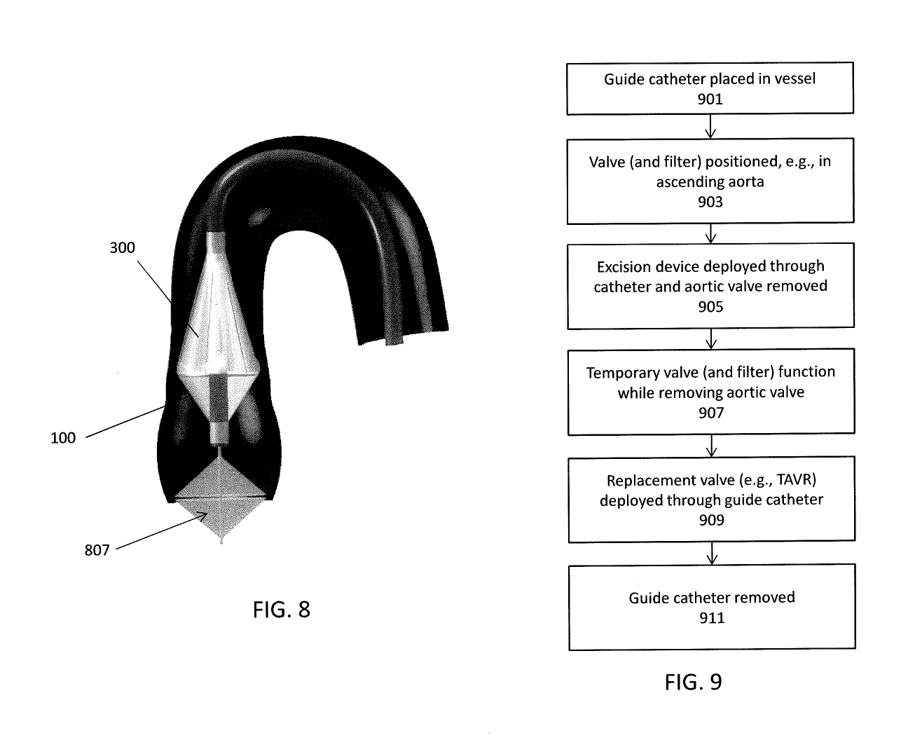

FIG. 8 illustrates one example of a method of using a temporary valve 100 (including an integrated filter 300) apparatus as described herein. In this example, the valve is shown inserted into the aortic arch and positioned in the between the aortic root and the brachiocephalic trunk. A valve removal apparatus 807 has been passed through the catheter and positioned on either side of the valve to be removed; it may then be operated through the catheter. The tissue may also be removed through the catheter.

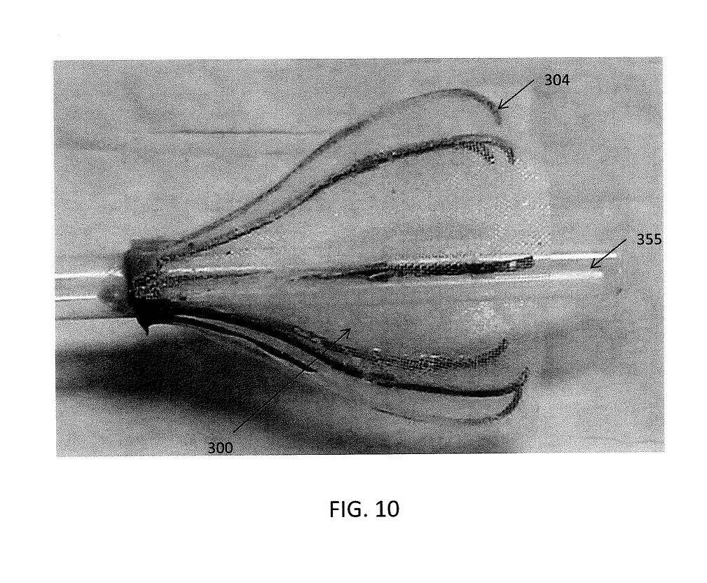

FIG. 9 schematically illustrates a general method of operating a temporary valve, which may optionally include a filter. The catheter (e.g., guide catheter) may first be positioned in the vessel 901, and the temporary valve, which may be fixedly or slideably attached to the catheter, may then be positioned in the desired location (e.g., the ascending aorta in this example) 903. The valve (and filter, when included) may be allowed to expand into position, e.g., by removing or expelling it from a sheath sliding on the catheter. Once in position, the valve may operate 907 while performing one or more additional procedures through the catheter, without disrupting operation of the temporary valve, including without disturbing its contact with the side of the vessel. For example, in FIG. 9, the optional step of passing an excision device (as illustrated in FIG. 8) through the catheter and removal of the aortic valve performed 905. In some variation, a replacement valve may then be positioned through the catheter, as described in optional step 909. Thereafter, the catheter (including the replacement valve may be removed) 911. Prior to removal, the temporary valve and, when used, filter, may be collapsed. For example, a sheath may be used to collapse the temporary valve and filter.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A temporary valve system, the system comprising:
a guide catheter having an inner diameter and an outer diameter and a proximal end and a distal end; and
a valve mounted around an outer distal portion of the guide catheter, the valve made of a thin and flexible material and comprising a conical distal section and a conical proximal section, the conical distal section of the valve tapering towards the distal end of the guide catheter and the conical proximal section of the valve tapering towards the proximal end of the guide catheter, the conical proximal section of the valve having a plurality of openings, the valve configured to adopt a collapsed configuration against the guide catheter when fluid flows over the valve from the distal end of the guide catheter to the proximal end of the guide catheter, the valve configured to adopt an expanded configuration when fluid flows over the valve from the proximal end of the guide catheter to the distal end of the guide catheter.

2. The temporary valve system of claim 1, further comprising a filter disposed on the guide catheter proximally to the distal section of the valve, the filter configured to remain in an open configuration when the valve is in either the expanded configuration or the collapsed configuration.

3. The temporary valve system of claim 1, wherein the valve comprises a one-piece construction with no rigid components.

4. The temporary valve system of claim 1, comprising a strutless valve wherein the conical distal section is unsupported by struts.

5. The temporary valve system of claim 2, wherein the filter comprises a support structure configured to support the filter in the open configuration.

6. The temporary valve system of claim 3, wherein the support structure comprises one or move of: a ring and a plurality of struts.

7. The temporary valve system of claim 3, wherein the support structure is made of a metal or shape memory metal.

8. The temporary valve system of claim 2, wherein the filter has a conical or bell-shaped shaped geometry.

9. The temporary valve system of claim 2, wherein the filter and valve are nested together.

10. The temporary valve system of claim 2, wherein the filter is slidably disposed on the guide catheter.

11. The temporary valve system of claim 2, further comprising a sheath that is slidably disposed over the guide catheter, valve, and filter.

12. The temporary valve system of claim 2, wherein the filter comprises one or more control wires that extend to the proximal end of the guide catheter, the one or more control wires configured to collapse and expand the filter.

13. The temporary valve system of claim 1, wherein the distal section of the valve is attached to the guide catheter with a distal end sleeve, the distal end sleeve having an inner diameter that matches the outer diameter of the guide catheter, and wherein the proximal section of the valve is attached to the guide catheter with a proximal end sleeve, the proximal end sleeve having an inner diameter that matches the outer diameter of the guide catheter.

14. The temporary valve system of claim 1, wherein the proximal section of the valve is longer than the distal section of the valve.

15. The temporary valve system of claim 1, wherein the proximal section of the valve is about twice as long as the distal section of the valve.

16. The temporary valve system of claim 1, wherein the proximal section of the valve is tapered at an angle between about 30 to 60 percent of the angle of the taper of the distal section of the valve.

17. The temporary valve system of claim 1, wherein the valve is slidably disposed on the guide catheter.

18. The temporary valve system of claim 2, wherein the valve and filter are supported by a common frame, the common frame comprising a first plurality of struts for supporting the filter and a second plurality of struts for supporting the valve.

19. The temporary valve system of claim 18, wherein the first plurality of struts for supporting the filter have free ends that are configured to open up to an expanded configuration, and wherein the second plurality of struts for supporting the valve have ends that remain proximate the guide catheter.

20. The temporary valve system of claim 2, wherein the valve and filter are concentrically disposed over the guide catheter.

21. A temporary valve system, the system comprising:
a guide catheter having an inner diameter and an outer diameter and a proximal end and a distal end;
a valve mounted around an outer distal portion of the guide catheter, the valve made of a thin and flexible material and comprising a conical distal section and a conical proximal section, wherein the conical distal section of the valve is unsupported by struts and tapers towards the distal end of the guide catheter, and wherein the conical proximal section of the valve tapers towards the proximal end of the guide catheter, the conical proximal section of the valve having a plurality of openings, the valve configured to adopt a collapsed configuration against the guide catheter when fluid flows over the valve from the distal end of the guide catheter to the proximal end of the guide catheter, the valve configured to adopt an expanded configuration when fluid flows over the valve from the proximal end of the guide catheter to the distal end of the guide catheter; and
a filter disposed on the guide catheter proximally to the distal section of the valve, the filter configured to remain in an open configuration when the valve is in either the expanded configuration or the collapsed configuration.

22. A method of providing a temporary valve to a blood vessel, the method comprising:
inserting a guide catheter into the blood vessel, the guide catheter having a valve mounted on an outer distal portion of the guide catheter, the valve made of a thin and flexible material and comprising a conical distal section and a conical proximal section, wherein the conical distal section is unsupported by struts and tapers towards the distal end of the guide catheter and wherein the conical proximal section of the valve tapers towards the proximal end of the guide catheter, the proximal section of the valve having a plurality of openings, the valve configured to adopt a collapsed configuration against the catheter when a fluid flows over the valve from the distal end of the guide catheter to the proximal end of the guide catheter, the valve configured to adopt an expanded configuration when the fluid flows over the valve from the proximal end of the guide catheter to the distal end of the guide catheter;
advancing the distal end of the guide catheter to the ascending aorta;
positioning the valve in the aorta; and
deploying the valve.

23. The method of claim 22, further comprising filtering blood passing past the guide catheter with a filter disposed on the guide catheter proximally to the distal section of the valve, the filter configured to remain in an open configuration when the valve is in either the expanded configuration or the collapsed configuration.

24. The method of claim 23, further comprising positioning the filter between the aortic root and brachiocephalic trunk.

25. The method of claim 22, wherein deploying the valve further comprises retracting a sleeve disposed over the guide catheter and the valve.

26. The method of claim 22, further comprising collapsing the valve by advancing a sleeve over the guide catheter and the valve.

27. The method of claim 22, wherein positioning the valve further comprises positioning the valve between the aortic root and the brachiocephalic trunk.

28. The method of claim 22, wherein positioning the valve further comprises positioning the valve in the descending aorta.

29. The method of claim 23, further comprising manipulating one or more control wires to deploy the filter.

30. The method of claim 22, further comprising deploying a valve excision device through the catheter and removing a cardiac valve.

31. The method of claim 22, further comprising deploying a replacement valve through the catheter.

32. A temporary filter system, the system comprising:
a guide catheter having an inner diameter and an outer diameter and a proximal end and a distal end;
a filter mounted around an outer distal portion of the guide catheter;
a sheath that is slidably disposed over the guide catheter and configured to slide distally to collapse the filter into a closed configuration and slide proximally to allow the filter to expand into an open configuration around the outer diameter of the guide catheter; and
a support structure configured to support the valve in the open configuration.

33. The temporary filter system of claim 32, wherein the support structure is a ring.

34. The temporary filter system of claim 32, wherein the support structure is a plurality of struts.

35. The temporary filter system of claim 32, wherein the support structure is made of a metal or shape memory metal.

36. The temporary filter system of claim 32, wherein the filter has a conical shaped geometry.

37. The temporary filter system of claim 32, wherein the filter has a bell shaped geometry.

38. The temporary filter system of claim 32, wherein the filter is slidably disposed on the guide catheter.

39. The temporary filter system of claim 32, wherein the filter comprises one or more control wires that extend to the proximal end of the guide catheter, the one or more control wires configured to collapse and expand the filter.
